(19) United States
(12) Patent Application Publication
(10) Pub. No.: US 2025/0054592 A1
(43) Pub. Date: Feb. 13, 2025

(54) SYSTEM AND METHOD FOR ANALYSIS OF ADVICE IN HEALTH MANAGEMENT ACTION

(71) Applicant: NEC Corporation, Tokyo (JP)

(72) Inventors: Kazuki SUZUKI, Tokyo (JP); Youichi KATO, Tokyo (JP); Shingo NAKAI, Tokyo (JP); Naoyuki NISHIO, Tokyo (JP); Keiko TANO, Tokyo (JP); Masaki IWAMOTO, Tokyo (JP)

(73) Assignee: NEC Corporation, Tokyo (JP)

(21) Appl. No.: 18/232,104

(22) Filed: Aug. 9, 2023

(51) Int. Cl. G16H 20/00; G06F 3/0482; G16H 40/67; G16H 50/70

(52) U.S. Cl. CPC G16H 20/00; G06F 3/0482; G16H 40/67; G16H 50/70

(57) ABSTRACT

A system according to an aspect of present disclosure acquires medical examination information about a result of medical examination of a user from a server and effort information about a healthy action from a user terminal, healthy action being an action engaged by the user; calculates an estimation value of an examination item of a medical examination of the user using a training model, the medical examination information and the effort information, the training model being a model trained on a relationship between a change in a value of an examination item of a medical examination and a healthy action by machine learning; generate advice on a healthy action based on a difference between a value of the examination item indicated in the medical examination information and the estimation value; converts the estimation value and the advice into the display signal; and transmits the display signal to the user terminal.

This application is based upon and claims the benefit of priority from Japanese Patent Application No. 2022-132327, filed on Aug. 23, 2022, the disclosure of which is incorporated herein in its entirety by reference.

### TECHNICAL FIELD

The present disclosure relates to a technique for assisting health management.

### BACKGROUND ART

There is a technique for simulating a health condition of a person. Reference literature (JP 2003-067499 A) discloses a medical examination system that allows an individual to be diagnosed about his/her health condition through a communication network. The system diagnoses a health condition of an individual using information about a physical element and an environmental element of the individual, and notifies the individual of a diagnosis result.

It is conceivable that a person who wants to grasp a health condition receives a medical examination at a hospital or the like. As a result of the medical examination, if the health condition is found to be unsatisfactory, the person is recommended to take a healthy action. Even when the person works on a healthy action, it is difficult for the person to know how much the health condition has been improved unless the person receives a medical examination again.

### SUMMARY

An object of the present disclosure is to provide a health management device or the like that enables a user to grasp a change in health condition.

A health management device according to an aspect of the present disclosure includes: one or more memories storing instructions; and one or more processors configured to execute the instructions to: acquire medical examination information indicating a result of a medical examination of a user and effort information indicating a healthy action that the user was engaged in; calculate, using a training model trained on a relationship between a change in a value of an examination item of a medical examination and a healthy action, an estimation value of an examination item of a medical examination of the user based on the medical examination information and the effort information; and present the calculated estimation value.

A health management method according to an aspect of the present disclosure includes: by a computer, acquiring medical examination information indicating a result of a medical examination of a user and effort information indicating a healthy action that the user was engaged in; calculating, using a training model trained on a relationship between a change in a value of an examination item of a medical examination and a healthy action, an estimation value of an examination item of a medical examination of the user based on the medical examination information and the effort information; and presenting the calculated estimation value.

A non-transitory recording medium according to an aspect of the present disclosure records a computer program for causing a computer to execute: acquiring medical examination information indicating a result of a medical examination of a user and effort information indicating a healthy action that the user was engaged in; calculating, using a training model trained on a relationship between a change in a value an examination item of a medical examination and a healthy action, an estimation value of an examination item of a medical examination of the user based on the medical examination information and the effort information; and presenting the calculated estimation value.

### BRIEF DESCRIPTION OF THE DRAWINGS

Exemplary features and advantages of the present disclosure will become apparent from the following detailed description when taken with the accompanying drawings in which:

FIG. 1 is a diagram schematically illustrating an example of a configuration of a health management device according to the first example embodiment;

FIG. 2 is a block diagram illustrating an example of a functional configuration of the health management device according to the first example embodiment;

FIG. 3 is a flowchart illustrating an example of an operation of the health management device according to the first example embodiment;

FIG. 4 is a block diagram illustrating an example of a functional configuration of a health management device according to the second example embodiment;

FIG. 5 is a diagram illustrating an example of an input form according to the second example embodiment;

FIG. 6 is a diagram illustrating another example of the input form according to the second example embodiment;

FIG. 7 is a diagram illustrating an example of estimation information according to the second example embodiment;

FIG. 8 is a diagram illustrating an example of similar user information according to the second example embodiment;

FIG. 9 is a flowchart for explaining an example of an operation of the health management device according to the second example embodiment;

FIG. 10 is a diagram illustrating an example of similar user information according to the fifth modification;

FIG. 11 is a block diagram illustrating an example of a functional configuration of a health management device according to the third example embodiment;

FIG. 12 is a flowchart for explaining an example of an operation of the health management device according to the third example embodiment; and

FIG. 13 is a block diagram illustrating an example of a hardware configuration of a computer device that implements the health management device according to the first, second, and third example embodiments of the present disclosure.

### EXAMPLE EMBODIMENT

Next, a detailed explanation will be given for a first example embodiment with reference to the drawings.

### First Example Embodiment

An outline of a health management device according to a first example embodiment will be described.

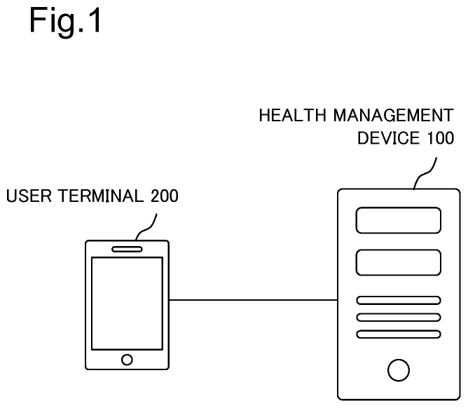

FIG. 1 is a diagram schematically illustrating an example of a configuration of a health management device 100. As illustrated in FIG. 1, for example, the health management device 100 is communicably connected to a user terminal 200 via a wireless or wired network.

The user terminal 200 is a terminal used by the user. The user terminal 200 may be a smartphone, a tablet terminal, a personal computer, or the like. The user terminal 200 receives an input from the user. For example, the user terminal 200 receives an input of information related to health management from the user. The user terminal 200 outputs information received from the health management device 100. The user terminal 200 includes a display or the like that performs a predetermined display based on a display signal supplied from the health management device 100.

Health management device 100 is a computer device. The health management device 100 may be, for example, a server. The health management device 100 acquires information input by the user from the user terminal 200. The health management device 100 presents information related to the health condition of the user based on the acquired information.

As described above, the health management device 100 is a device that provides the user with information related to the health condition of the user.

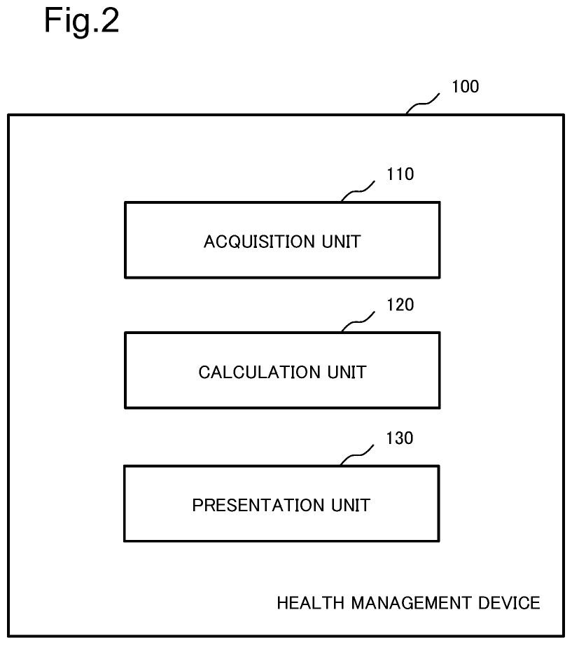

Next, an example of a functional configuration of the health management device 100 will be described. FIG. 2 is a block diagram illustrating an example of a functional configuration of the health management device 100. As illustrated in FIG. 2, the health management device 100 includes an acquisition unit 110, a calculation unit 120, and a presentation unit 130.

The acquisition unit 110 acquires the medical examination information. The medical examination information is information indicating a result of the medical examination of the user. For example, the medical examination information indicates a result of a medical examination that the user actually receives in an institution such as a hospital. That is, the medical examination information includes information about the value of the examination item in the medical examination. For example, the user inputs the medical examination information to the user terminal 200. Then, the acquisition unit 110 may acquire the medical examination information from the user terminal 200. The present disclosure is not limited to this example, and the acquisition unit 110 may acquire the medical examination information from, for example, a server (not illustrated) of an institution having the medical examination information.

The acquisition unit 110 acquires effort information. The effort information is information indicating a healthy action that the user was engaged in. The healthy action is an action for the purpose of maintaining, recovering, improving, and the like of health. Examples of the healthy action include exercise, diet, and sleep. For example, the user inputs the effort information to the user terminal 200. Then, the acquisition unit 110 may acquire the effort information from the user terminal 200. The present disclosure is not limited to this example, and the acquisition unit 110 may acquire, for example, measured information as effort information from a device that measures an activity amount.

The timing at which the acquisition unit 110 acquires the medical examination information may be different from the timing at which the acquisition unit acquires the effort information.

In this manner, the acquisition unit 110 acquires the medical examination information indicating the result of the medical examination of the user and the effort information indicating the healthy action that the user was engaged in.

The calculation unit 120 calculates an estimation value for each examination item of the medical examination of the user. The examination item indicates content to be tested in the medical examination. For example, the examination item may include various values of a test performed for a biological function such as liver function, lipid metabolism, and diabetes. In the case of a test related to liver function, the examination items are, for example, values of glutamate oxaloacetate transaminase (GOT), glutamic pyruvic transaminase (GPT), and γ-glutamyl transpeptidase (γ-GPT).

The calculation unit 120 calculates an estimation value based on the acquired medical examination information and effort information. At this time, the calculation unit 120 calculates the estimation value using the training model. For example, the training model is a model that was machine-trained on the relationship between the change in the value of the examination item of the medical examination and the healthy action. The calculation unit 120 calculates an estimation value of the value of the examination item by inputting the acquired medical examination information and effort information to such a training model. That is, the estimation value is an estimation value of the examination item in a case where the user performs the healthy action indicated in the effort information.

In this manner, the calculation unit 120 calculates the estimation value for each examination item of the medical examination of the user based on the medical examination information and the effort information using the training model that was trained on the relationship between the change in the value of the examination item of the medical examination and the healthy action. The calculation unit 120 is an example of a calculation means.

The presentation unit 130 presents the estimation value. For example, the presentation unit 130 presents the estimation value by transmitting information including the estimation value to the user terminal 200. The information including the estimation value is displayed, for example, on a display included in the user terminal 200. In other words, the calculated estimation value is converted into the display signal and transmitted the display signal to user terminal.

In this manner, the presentation unit 130 presents the calculated estimation value. Presentation unit 130 is an example of a presentation means.

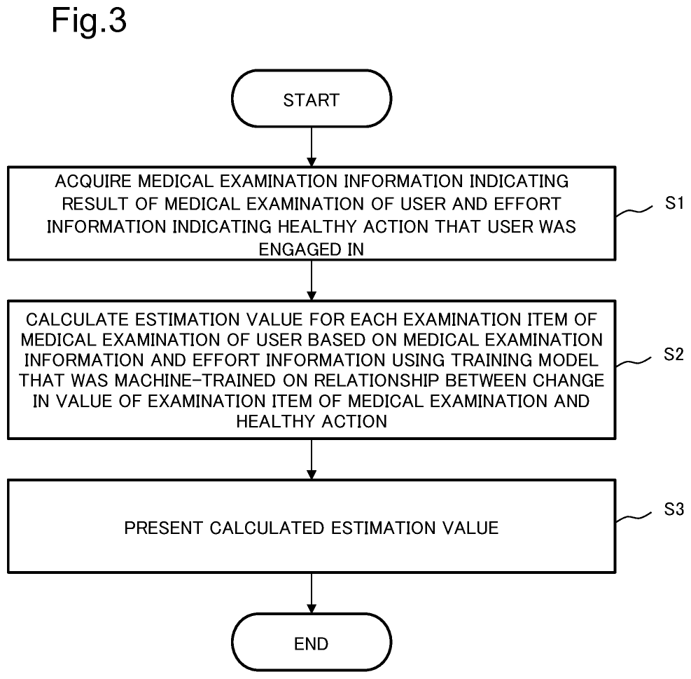

Next, an example of the operation of the health management device 100 will be described with reference to FIG. 3. Note that, in the present disclosure, each step of the flowchart is represented using a number assigned to each step, such as “S1”.

FIG. 3 is a flowchart illustrating an example of the operation of the health management device 100. The acquisition unit 110 acquires the medical examination information indicating the result of the medical examination of the user and the effort information indicating the healthy action that the user was engaged in (S1). The calculation unit 120 calculates the estimation value for each examination item of the medical examination of the user based on the medical examination information and the effort information using the training model that was trained on the relationship between the change in the value of the examination item of the medical examination and the healthy action (S2). Then, the presentation unit 130 presents the calculated estimation value (S3).

As described above, the health management device 100 according to the first example embodiment acquires the medical examination information indicating the result of the medical examination of the user and the effort information indicating the healthy action that the user was engaged in. The health management device 100 calculates an estimation value for each examination item of the medical examination of the user based on the medical examination information and the effort information using a training model that was trained on the relationship between the change in the value of the examination item of the medical examination and the healthy action. Then, the health management device 100 presents the calculated estimation value.

As a result, the health management device 100 can provide the user with the estimation value for each examination item of the medical examination. Therefore, the user can grasp how much the health condition has changed as compared with the result of the medical examination. That is, the health management device 100 allows the user to grasp the change in the health condition. Furthermore, the user can, for example, decide on the necessary actions based on the provided estimation value. The necessary actions may include, for example, exercising, adopting a diet, consulting with healthcare professionals. In this way, the health management device 100 can assist the user in decision making.

### Second Example Embodiment

Next, a health management device according to the second example embodiment will be described. In a second example embodiment, a further example of the health management device described in the first example embodiment will be described. Part of the description of content overlapping with that of the first example embodiment will be omitted.

### Details of Health Management Device 100

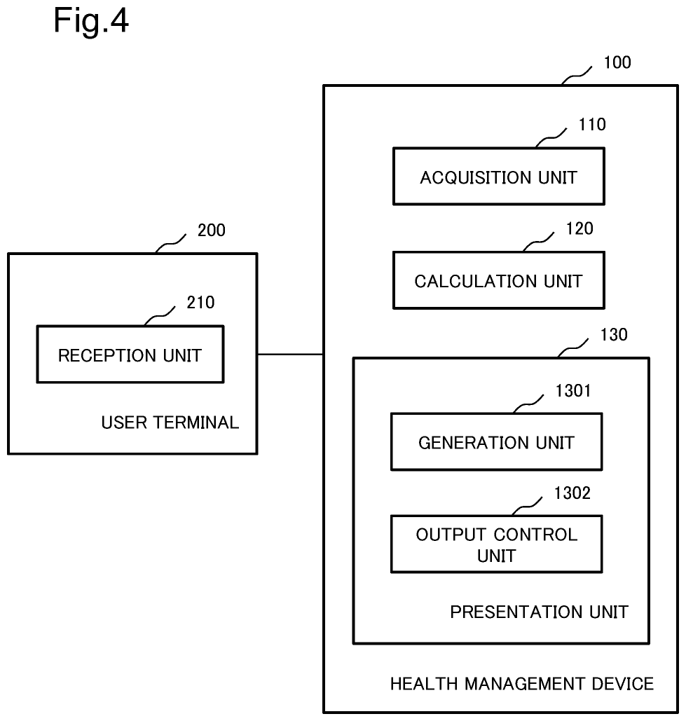

FIG. 4 is a block diagram illustrating an example of a functional configuration of the health management device 100. As illustrated in FIG. 4, the health management device 100 includes the acquisition unit 110, the calculation unit 120, and the presentation unit 130.

The user terminal 200 includes a reception unit 210. The reception unit 210 accepts an input from a user who uses the user terminal 200. The reception unit 210 receives an input of medical examination information from the user. For example, an input form for receiving an input of the medical examination information is displayed on a display included in the user terminal 200. For example, a display control unit (not illustrated) of the health management device 100 may display the input form on the user terminal 200. The reception unit 210 receives an input of the medical examination information through the input form. The reception unit 210 receives an input of a value for each examination item of the medical examination received by the user. The examination items include, but are not limited to, various values of tests performed on biological functions such as liver function, lipid metabolism, and diabetes. For example, the examination item may include values of tests regarding a circulatory system and a respiratory system, or may include a value of a hematological test. The examination item may include information such as a height, a weight, an abdominal circumference, and a body mass index (BMI).

In this manner, the reception unit 210 receives the input of the medical examination information from the user. The reception unit 210 transmits the input medical examination information to the health management device 100. The acquisition unit 110 acquires the transmitted medical examination information.

The method of acquiring the medical examination information is not limited to this example. For example, the reception unit 210 receives an input of identification information from the user. The identification information is information for identifying the user. Reception unit 210 transmits the identification information to health management device 100. The acquisition unit 110 acquires identification information. Then, the acquisition unit 110 may acquire the medical examination information from an external server (not illustrated) communicably connected to the health management device 100 based on the identification information. At this time, the external server has, for example, a database in which the medical examination information about each of the plurality of persons and the identification information are associated with each other. The external server may be, for example, a device included in an institution that performs a medical examination. The acquisition unit 110 may acquire the medical examination information related to the acquired identification information from the external server.

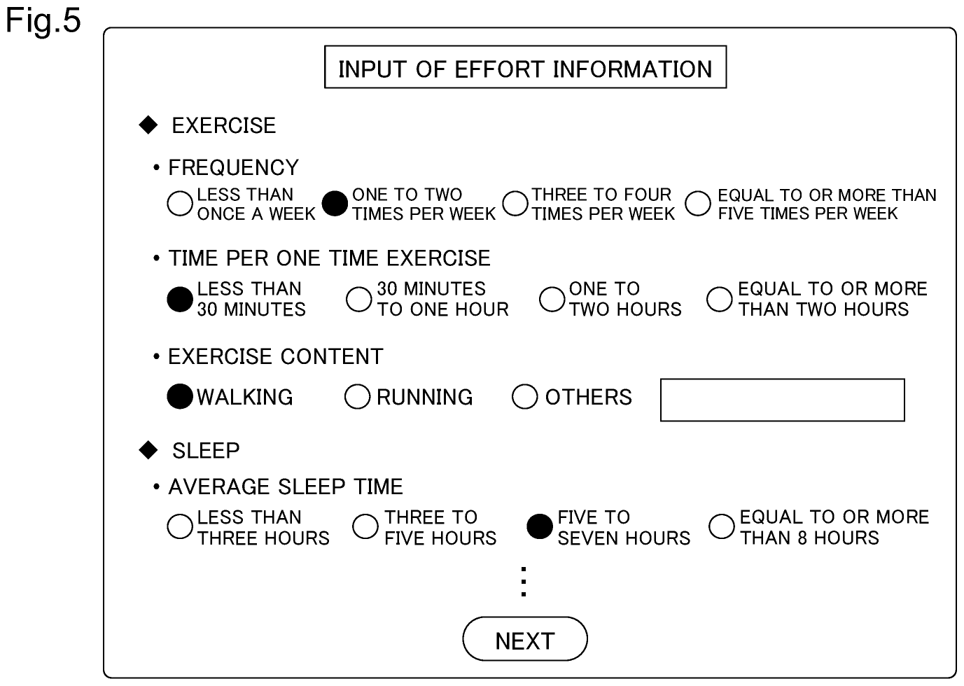

The reception unit 210 receives input of effort information from the user. For example, an input form for receiving input of the effort information is displayed on a display of the user terminal 200. FIG. 5 is a diagram illustrating an example of an input form. More specifically, FIG. 5 is an example of an input form for receiving the input of effort information. The reception unit 210 receives the input of the effort information through, for example, an input form as illustrated in FIG. 5. The effort information indicates a healthy action that the user was engaged in. In the example of FIG. 5, an input regarding an action that the user was engaged in is received regarding exercise and sleep. For example, in the case of exercise, the user inputs frequency, time per exercise, and exercise content. In this manner, the effort information may include information about the average of the healthy actions that the user was engaged in.

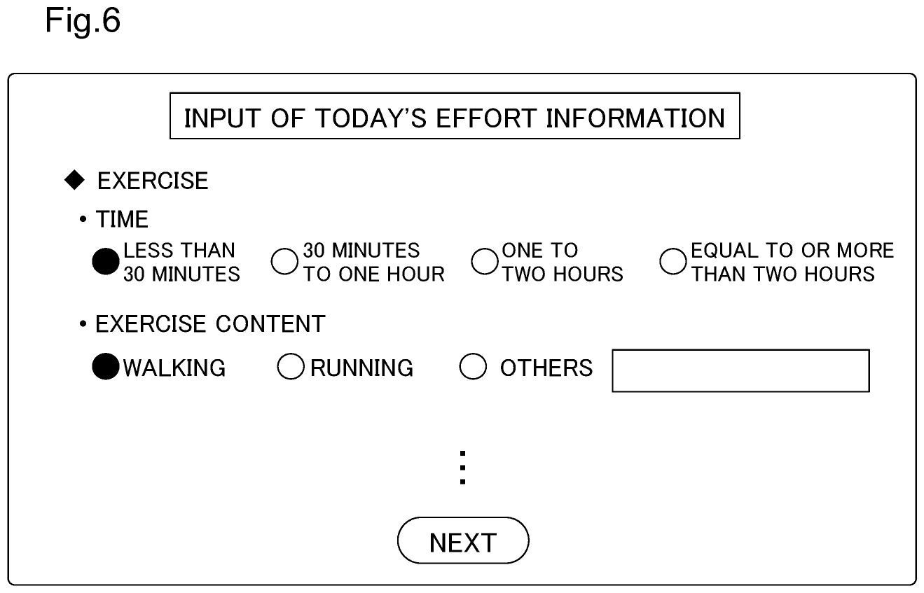

The input effort information is not limited to this example. FIG. 6 is a diagram illustrating another example of the input form. More specifically, FIG. 6 is a diagram illustrating another example of an input form for receiving input of effort information. In the example of FIG. 6, “input of today's effort information” is urged. That is, the reception unit 210 may receive the input of the effort information for each healthy action that the user was engaged in. For example, when the user walked for less than 30 minutes today, as illustrated in FIG. 6, reception unit 210 receives selection of an option of “less than 30 minutes” and an option of “walking”. In the case of a healthy action related to a meal, the reception unit 210 may receive the input of the content of a meal of the user for each meal. That is, the reception unit 210 may receive a plurality of times of input of the effort information.

In this manner, the reception unit 210 receives the input of the effort information from the user. Reception unit 210 transmits the input effort information to health management device 100. Acquisition unit 110 acquires the transmitted effort information.

The calculation unit 120 calculates an estimation value for each examination item of the medical examination based on the acquired medical examination information and effort information. At this time, the calculation unit 120 calculates the estimation value using the training model. The training model is, for example, a model that was machine-trained on the relationship between the change in the value of the examination item of the medical examination and the healthy action. In this case, the calculation unit 120 inputs the acquired medical examination information and effort information to the training model. Then, the value of the examination item of the medical examination is output by the training model. The calculation unit 120 calculates the output value as an estimation value of the examination item for the user. The training model may be, for example, a linear model such as a generalized estimation equation, or a model trained by deep learning or the like using a neural network. The example of the training model is not limited to this example. The training model may be a model in which the medical examination information and the effort information are used as explanatory variables and the value of the examination item of the medical examination included in the medical examination information is used as an objective variable.

Presentation unit 130 includes a generation unit 1301 and an output control unit 1302. The generation unit 1301 generates estimation information. The estimation information is information indicating the estimated health condition of the user. The estimation information includes the calculated estimation value. The output control unit 1302 causes the user terminal 200 to output the information generated by the generation unit 1301. Specifically, the output control unit 1302 transmits the estimation information to the user terminal 200. The estimation information is displayed, for example, on a display included in the user terminal 200.

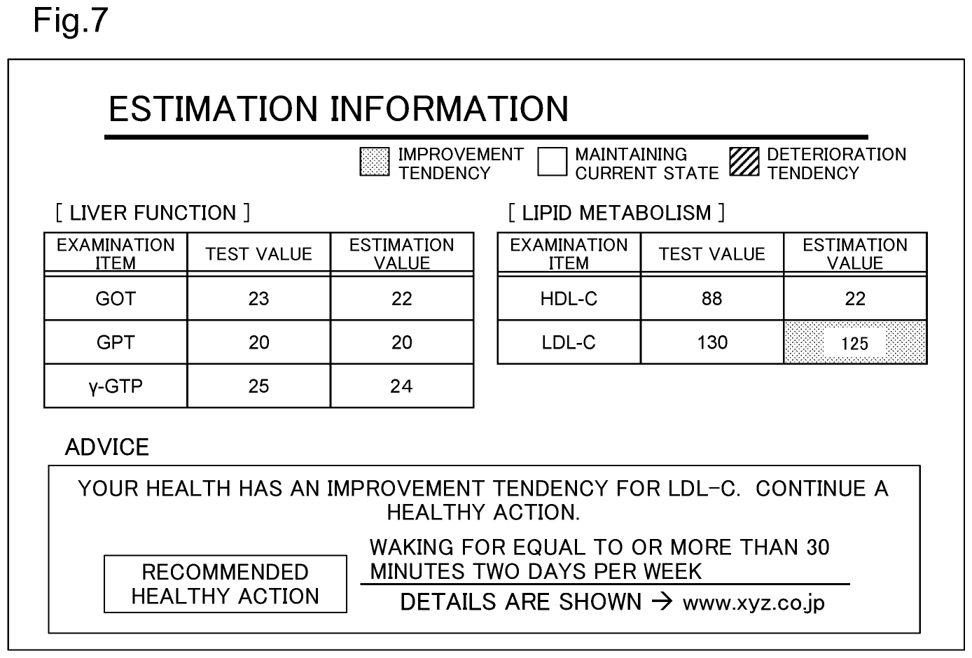

The generation unit 1301 generates, for example, estimation information including a value of the examination item indicated in the medical examination information and an estimation value. FIG. 7 is a diagram illustrating an example of the estimation information. In the example of FIG. 7, information in which the test value and the estimation value are associated with each other for each examination item is illustrated. The unit of each value is omitted. The test value is a value of an examination item of a medical examination actually received by the user. That is, the test value is a value of the examination item indicated in the medical examination information.

In the example of FIG. 7, the test value is “25” and the estimation value is “24” for the examination item “γ-GTP”. The tendency of the change in the estimation value may be indicated in the estimation information. The tendency of the change in the estimation value is indicated by the difference between the test value and the estimation value. In the example of FIG. 7, for the examination item “LDL-C”, it is indicated that the test value is “130” and the estimation value is “125”. LDL-C represents low density lipoproteinn-cholesterol. In this example, the estimation value of LDL-C is lower than the test value. That is, the estimation value has an improvement tendency. Then, the column of the estimation value “125” having an improvement tendency is emphasized. In this manner, in a case where the estimation value has an improvement tendency, the generation unit 1301 may generate estimation information indicating that there is an improvement tendency. Similarly, the generation unit 1301 may generate estimation information indicating a tendency of a change in each estimation value even in a case where the estimation value is maintained as it is or has a deterioration tendency. In the example of FIG. 7, patterns related to a case where the estimation value tends to be improved compared to the test value, a case where the current state is maintained, and a case where the estimation value tends to be deteriorated are illustrated.

The estimation information may include advice on a healthy action. In the example of FIG. 7, a comment such as “your health has an improvement tendency for LDL-C. Let's continue the healthy action” is indicated according to the trend of change in the estimation value. In this manner, the generation unit 1301 may generate a comment according to the difference between the test value and the estimation value. That is, the presentation unit 130 may present the advice on the healthy action based on the difference between the value of the examination item indicated in the medical examination information and the estimation value. In other words, the advice is converted into the display signal and transmitted the display signal to user terminal.

The advice may include information indicating healthy action recommended to the user. In the example of FIG. 7, “walking for equal to or more than 30 minutes 2 days per week” is indicated as the“recommended healthy action”. In this manner, the generation unit 1301 may determine a healthy action recommended to the user. The healthy action recommended to the user may indicate at least one of an exercise method and content of a meal. Furthermore, the healthy action recommended to the user may include a sleeping method and the like. At this time, for example, a healthy action according to the tendency of the change in the estimation value is determined in advance. The generation unit 1301 identifies a healthy action related to the tendency of the change in the estimation value of the user. Then, the generation unit 1301 generates advice including information indicating the identified healthy action.

Furthermore, in the example of FIG. 7, a uniform resource locator (URL) indicating details of the recommended healthy action is illustrated. The URL is, for example, information indicating a web page describing a walking method. The present disclosure is not limited to this example, and book information indicating details of the recommended healthy action may be indicated. In this manner, the generation unit 1301 may generate advice indicating information about the medium in which the healthy action is indicated. That is, the presentation unit 130 may present advice indicating information about a medium indicating at least one of a method of exercise as a healthy action or content of a meal as a healthy action.

The generation unit 1301 may generate similar user information indicating information about another user similar to the user. Another user similar to the user is referred to as a similar user. The similar user is, for example, a user whose result of the medical examination is similar to that of the user. The generation unit 1301 identifies a similar user based on the medical examination information about the user and the medical examination information about another user. At this time, the generation unit 1301 compares the medical examination information about the user with the medical examination information about another user for each examination item. Then, in a case where the number of examination items for which the difference between the test values is within the predetermined range is equal to or more than the threshold value, the generation unit 1301 identifies the other user as a similar user. Then, the generation unit 1301 generates similar user information indicating the healthy action performed by the similar user. The output control unit 1302 transmits the similar user information to the user terminal 200 to cause the user terminal 200 to output the similar user information.

The method of identifying the similar user is not limited to this example. For example, the reference range is set for each examination item. An examination item whose test value deviates from the reference range is diagnosed as abnormal. In a case where there is an abnormal examination item in the user, the generation unit 1301 may identify another user in which an examination item same as the examination item is abnormal as the similar user. Then, the generation unit 1301 may generate the similar user information in such a way as to include a change between the test value and the estimation value of the examination item determined to be abnormal.

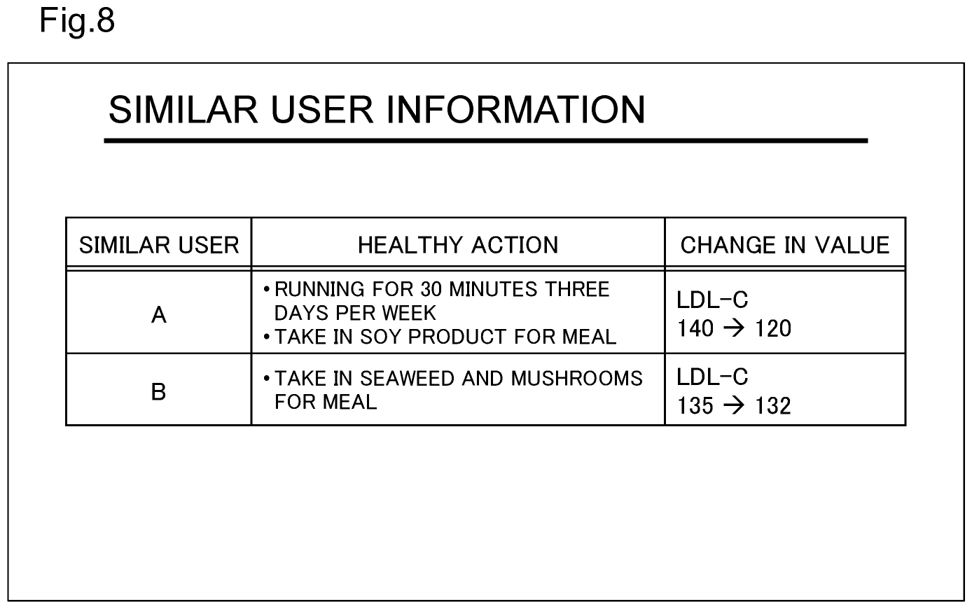

FIG. 8 is a diagram illustrating an example of similar user information. In the example of FIG. 8, the healthy action performed by the similar user and the change between the test value and the estimation value of the abnormal examination item are illustrated. For example, the figure illustrates a similar user “B” is “taking in seaweed and mushrooms for the meal” as a healthy action. Regarding the similar user “B”, it is indicated that the examination item “LDL-C” changes from the test value “135” to the estimation value “132”.

In this manner, in a case where there is another user whose result of the medical examination is similar to that of the user, the presentation unit 130 may present the healthy action performed by the another user. By presenting the healthy action of another user, the user can grasp what kind of healthy action another person is performing. Therefore, the user can refer to the healthy action of another person.

### Operation Example of Health Management Device 100

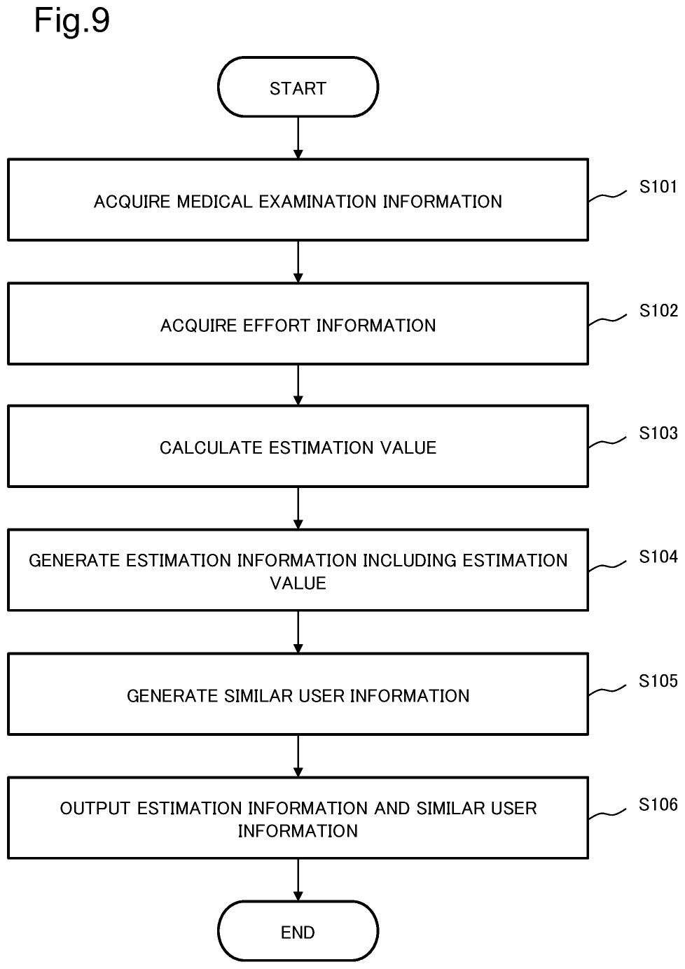

Next, an example of the operation of the health management device 100 will be described with reference to FIG. 9.

FIG. 9 is a flowchart illustrating an example of the operation of the health management device 100. First, the user terminal 200 displays an input form. The reception unit 210 accepts input of information about a subject. Then, the reception unit 210 transmits the received information about a subject to the health management device 100.

The acquisition unit 110 acquires the medical examination information from the user terminal 200 (S101). The acquisition unit 110 acquires the effort information from the user terminal 200 (S102). The calculation unit 120 calculates an estimation value based on the acquired medical examination information and effort information (S103).

The generation unit 1301 generates estimation information including the calculated estimation value (S104). At this time, the estimation information may include advice on a healthy action based on a difference between the test value and the estimation value. The generation unit 1301 generates similar user information including the healthy action performed by the similar user (S105). Then, the output control unit 1302 causes the user terminal 200 to output the estimation information and the similar user information (S106).

The operation of the health management device 100 is not limited to this example. For example, in a case where the medical examination information is acquired in advance, such as after the estimation information is generated once, the health management device 100 may start the processing from S102. In this case, for example, the calculation unit 120 may calculate the estimation value based on the previously acquired medical examination information, the accumulated effort information, and the newly acquired effort information.

As described above, the health management device 100 according to the second example embodiment acquires the medical examination information indicating the result of the medical examination of the user and the effort information indicating the healthy action that the user was engaged in. The health management device 100 calculates an estimation value for each examination item of the medical examination of the user based on the medical examination information and the effort information using a training model that was trained on the relationship between the change in the value of the examination item of the medical examination and the healthy action. Then, the health management device 100 presents the calculated estimation value.

As a result, the health management device 100 can provide the user with the estimation value for each examination item of the medical examination. Therefore, the user can grasp how much the health condition has changed as compared with the result of the medical examination. That is, the health management device 100 allows the user to grasp the change in the health condition. Since the user can grasp the change in the health condition, the user can achieve the result of the healthy action. That is, the health management device 100 can suppress a decrease in motivation for the user's healthy action.

In a case where there is another user whose result of the medical examination is similar to that of the user, the health management device 100 may present a healthy action performed by the other user. As a result, the health management device 100 can notify the user of the healthy action performed by another person. Therefore, the user can refer to the healthy action performed by another person similar to the user.

The health management device 100 may present advice on a healthy action based on a difference between the value of the examination item indicated in the medical examination information and the estimation value. As a result, the health management device 100 can notify the user of a healthy action suitable for the user.

### First Modification

In the above-described example embodiment, an example in which the health management device 100 is communicably connected to the user terminal 200 is described. On the other hand, the configuration of the health management device 100 is not limited to this example. For example, the user terminal 200 may include the acquisition unit 110, the calculation unit 120, and the presentation unit 130. In this case, the user terminal 200 is communicably connected to the external server. Then, the external server stores the training model for calculating the estimation value and the medical examination information and the estimation value of each of the plurality of users.

### Second Modification

In the above-described example embodiment, an example is described in which the acquisition unit 110 acquires the effort information by the user inputting the input form. On the other hand, the method for acquiring the effort information is not limited to this example. For example, there is a case where history information such as an exercise history, a sleep history, and a meal history of the user is acquired by an application installed in the user terminal 200. In such a case, the acquisition unit 110 may acquire the history information acquired by the application as the effort information.

An exercise history, a sleep history, and the like of the user may be acquired by the wearable terminal. In this case, the acquisition unit 110 may acquire the exercise history, the sleep history, and the like from the wearable terminal as the effort information.

### Third Modification

The advice presented by the presentation unit 130 may include various pieces of information. For example, the advice may include information about a benefit and welfare system of the place of employment of the user. Specifically, the advice may indicate a gym, a meal delivery service, a fitness machine sales service, and the like that can be used through the benefit and welfare system. That is, the advice may include information indicating a benefit and welfare system related to healthy action.

In this case, the acquisition unit 110 acquires information indicating the place of employment of the user. Then, the generation unit 1301 acquires information about the benefit and welfare system of the place of employment of the user. At this time, a database in which information indicating the benefit and welfare system is associated in advance for each place of employment is stored in the health management device 100 or an external server communicably connected to the health management device 100. The generation unit 1301 acquires, for example, information indicating the benefit and welfare system from the database. Then, the generation unit 1301 generates the estimation information indicating the advice including the information indicating the benefit and welfare system. The output control unit 1302 performs control to output the estimation information to the user terminal 200.

In this manner, the acquisition unit 110 may acquire the information indicating the place of employment of the user. Then, the presentation unit 130 may present advice including information indicating a benefit and welfare system related to the healthy action, the benefit and welfare system being a benefit and welfare system of the place of employment of the user.

### Fourth Modification

The estimation information may indicate a future health condition together with the estimation value. Specifically, the estimation information may indicate information about a disease at risk of onset, or may indicate information indicating that there is no disease at risk of onset. For example, when the estimation value of LDL-C is higher than a predetermined reference value, a comment such as “myocardial infarction and cerebral infarction are likely to occur in the future” may be included in the estimation information.

### Fifth Modification

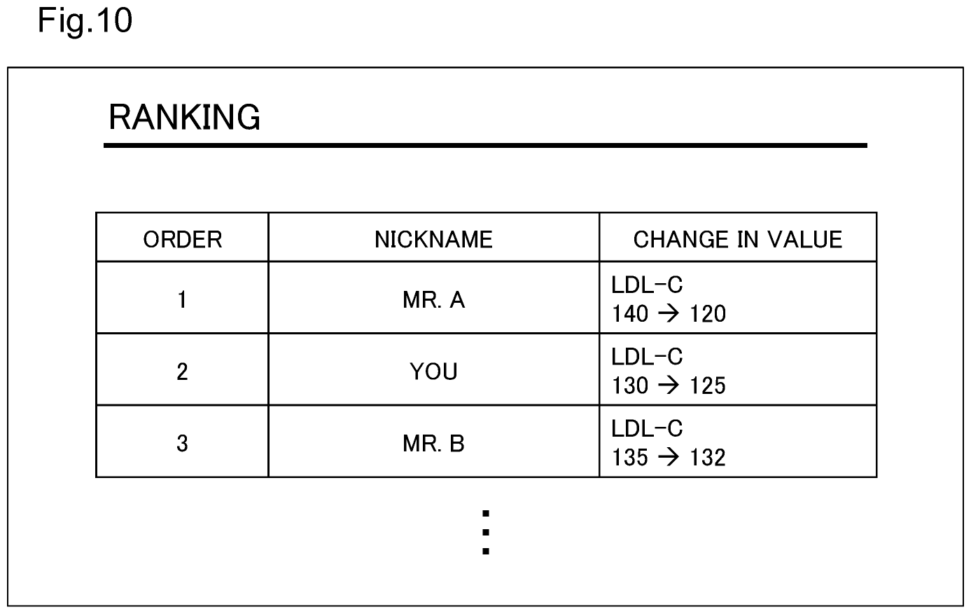

The similar user information may include information obtained by comparing the user with another user. FIG. 10 is a diagram illustrating an example of similar user information about the fifth modification. In the example of FIG. 10, the ranking of the user and the similar users is illustrated. The ranking is a list ranked based on a difference between the test value and the estimation value related to the examination item “LDL-C”. In this example, ranks 1 and 3 are information about similar users, and rank 2 is information about users. For example, for the similar user of the rank 1, a difference between the test value and the estimation value of “LDL-C” is 20, and for the user of the rank 2, a difference between the test value and the estimation value of “LDL-C” is 5. In this manner, the list may be information ranked in descending order of the degree of improvement regarding the specific examination item.

At this time, the calculation unit 120 calculates estimation values of a plurality of users in advance. Then, the generation unit 1301 calculates a difference between the test value and the estimation value for the user and the similar user. That is, the generation unit 1301 calculates a difference between the value of the examination item indicated in the medical examination information and the estimation value for each of two or more users having similar medical examination results.

In addition, the generation unit 1301 generates a list in which users and similar users are ranked according to the difference. The output control unit 1302 performs control to output similar user information including the generated list to the user terminal 200.

In this manner, the health management device 100 calculates estimation values of a plurality of users. The health management device 100 calculates a difference between the value of the examination item indicated in the medical examination information and the estimation value for each of two or more users having similar medical examination results to generate a list in which the two or more users are ranked according to the difference. Then, the health management device 100 presents the generated list. As a result, the health management device 100 can indicate the relative position of the user with respect to the improvement degree of the health condition as compared with other users.

### Sixth Modification

The health management device 100 may further present information indicating the medical examination information and the effort information about the user. That is, the health management device 100 may present the accumulated medical examination information and effort information to the user.

The generation unit 1301 generates accumulated information indicating the accumulated medical examination information and the effort information. At this time, the accumulated information may also include information about the estimation value at the time when each effort information is input. Then, the output control unit 1302 performs control to output the accumulated information to the user terminal 200. As a result, the health management device 100 can allow the user to grasp again the healthy action that the user has been engaged in so far.

Such accumulated information can be useful data in the relationship between the healthy action and the value of the examination item. The user can also sell the accumulated information to a company that conducts a health-related business.

### Third Example Embodiment

Next, a health management device according to the third example embodiment will be described. In a third example embodiment, an example of calculating an estimation value using further information in addition to effort information will be described. Part of the description of content overlapping with that of the first and second example embodiments will be omitted.

### Details of Health Management Device 101

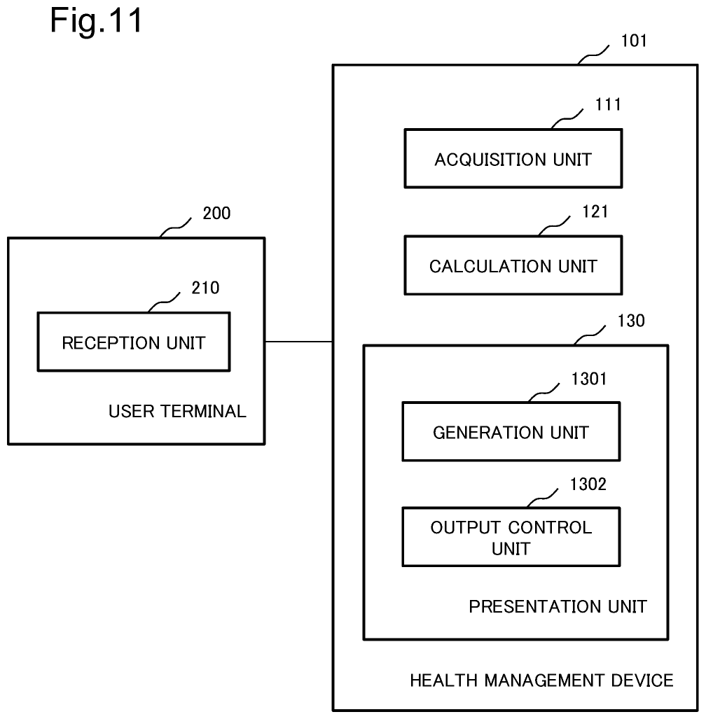

FIG. 11 is a block diagram illustrating a functional configuration of a health management device 101. As illustrated in FIG. 11, the health management device 101 is communicably connected to the user terminal 200 via a wired or wireless network.

In the third example embodiment, the health management device 101 acquires further information in addition to the medical examination information and the effort information. Examples of the further information include biometric information measured by various measurement instruments. The measurement instrument is, for example, a body composition meter and a sphygmomanometer. That is, a specific example of the further information is basic information about the user. The basic information is information about the body composition of the user. For example, the basic information includes a height, a weight, a body fat percentage, a BMI, a muscle mass, a basal metabolic rate, and the like of the user. The basic information may be information measured by a body composition meter. A specific example of the information is blood pressure information. The blood pressure information indicates the blood pressure of the user measured by the sphygmomanometer.

An example of the information is genetic information about the user. The genetic information may be, for example, information encoded as a base sequence of deoxyribonucleic acid (DNA), information indicating information about an amino acid sequence of a protein, and the like. The present disclosure is not limited to this example, and the genetic information may be information indicating a disease of a relative of the user. Specifically, in a case where the user's grandfather has developed cancer, the genetic information may include information indicating that the grandfather has developed cancer.

As illustrated in FIG. 11, the health management device 101 includes an acquisition unit 111, a calculation unit 121, and a presentation unit 130.

The acquisition unit 111 acquires biometric information measured by the measurement instrument. Specifically, the acquisition unit 111 may acquire the basic information about the user from the body composition meter used by the user. At this time, the body composition meter and the health management device 101 are communicably connected. For example, the acquisition unit 111 acquires the basic information in response to the use of the body composition meter by the user.

In addition, the acquisition unit 111 may acquire the blood pressure information about the user from the sphygmomanometer used by the user. At this time, the sphygmomanometer and the health management device 101 are communicably connected. For example, the acquisition unit 111 acquires the blood pressure information in response to the use of the sphygmomanometer by the user.

The method by which the acquisition unit 111 acquires the biometric information measured by the measurement instrument is not limited to this example. For example, the acquisition unit 111 may acquire the biometric information from the user terminal 200. In this case, the user terminal 200 and the measurement instrument are communicably connected. Then, the user terminal 200 acquires the biometric information from the measurement instrument in response to the measurement of the biometric information about the user by the measurement instrument. Then, the acquisition unit 111 acquires the biometric information from the user terminal 200.

The method by which the user terminal 200 acquires the biometric information may be another method. For example, the user terminal 200 may acquire the biometric information based on the identification information output from the measurement instrument. The identification information is information associated with the biometric information measured by the measurement instrument. That is, the identification information is information for accessing the biometric information. The identification information may be predetermined character information or a two-dimensional barcode. At this time, the identification information may be displayed on a display included in the measurement instrument, or may be printed on paper output by the measurement instrument. The user terminal 200 receives an input of identification information by a user's operation. For example, in a case where the identification information is character information, information in which the identification information and the biometric information are associated with each other is stored in an external device such as a server. When the user inputs the identification information to the user terminal 200, the user terminal 200 acquires the biometric information associated with the input identification information. On the other hand, in a case where the identification information is a two-dimensional barcode, for example, the two-dimensional barcode indicates biometric information. When the user photographs the two-dimensional barcode with the camera included in the user terminal 200, the biometric information indicated in the two-dimensional barcode is input to the user terminal 200.

In this manner, the acquisition unit 111 acquires the biometric information about the user that is information measured by the measurement instrument used by the user.

The measurement instruments such as a body composition meter and a sphygmomanometer may be installed in stores such as a drug store and a gym. The acquisition unit 111 may acquire the biometric information about the user from a measurement instrument of the biometric information installed in such a store. That is, the acquisition unit 111 may acquire the biometric information about the user measured by the measurement instrument installed in the store. As a result, the health management device 101 can use the biometric information even if the user does not possess the measurement instrument.

The acquisition unit 111 may acquire genetic information about the user. The genetic information is stored in a storage device included in the user terminal 200 in advance, for example. In this case, the acquisition unit 111 acquires the genetic information from the user terminal 200. The acquisition unit 111 may acquire the genetic information from an external server. In this case, the external server stores the genetic information and the identification information for identifying the user in association with each other. The acquisition unit 111 acquires identification information for identifying the user from the user terminal 200. Then, the acquisition unit 111 may acquire the genetic information from the external server using the identification information.

The calculation unit 121 further uses biometric information or genetic information to calculate an estimation value. When the biometric information is used, the training model is a model that was machine-trained on the relationship between the change in the value of the examination item of the medical examination, the healthy action, and the basic information. At this time, for example, the calculation unit 121 calculates the estimation value by inputting the acquired medical examination information, effort information, and biometric information to the training model. In this manner, the calculation unit 121 may calculate the estimation value of the user based on the medical examination information, the effort information, and the basic information using the training model that was trained on the relationship between the change in the value of the examination item of the medical examination, the healthy action, and the basic information.

In a case where the genetic information is used, the training model is a model that was machine-trained on the relationship between the change in the value of the examination item of the medical examination, the healthy action, and the genetic information. At this time, for example, the calculation unit 121 calculates the estimation value by inputting the acquired medical examination information, effort information, and genetic information to the training model. The calculation unit 121 may calculate the estimation value using both the biometric information and the genetic information.

### Operation Example of Health Management Device 101

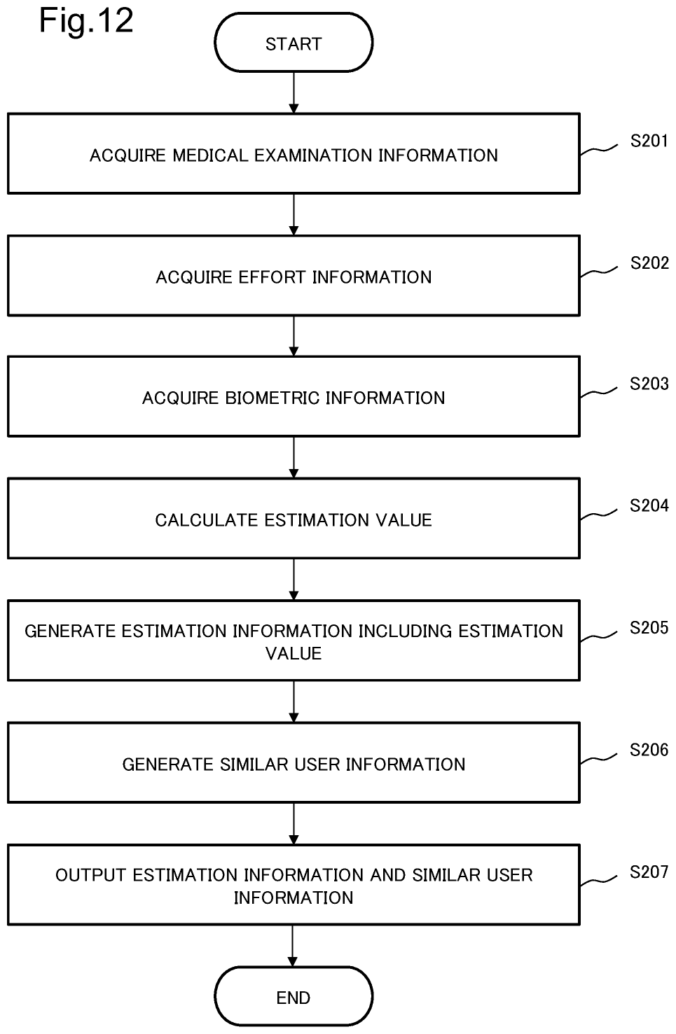

Next, an example of the operation of the health management device 101 will be described with reference to FIG. 12. In the present operation example, an example in which the health management device 101 uses biometric information measured by a measurement instrument will be described.

FIG. 12 is a flowchart illustrating an example of the operation of the health management device 101. The processing of S201 and S202 is similar to the processing of S101 and S102 of FIG. 9. The acquisition unit 111 acquires the biometric information about the user from the user terminal 200 (S203). Then, the calculation unit 121 calculates an estimation value based on the acquired medical examination information, effort information, and biometric information (S204).

The processing of S205 to S207 is similar to the processing of S104 to S106.

The operation of the health management device 101 is not limited to this example. For example, the acquisition unit 111 may acquire the biometric information together with the effort information. The health management device 101 may acquire genetic information before the processing of S204. Then, in the processing of S204, the health management device 101 may further calculate an estimation value in consideration of the genetic information. Various diseases may involve genetic factors. The health management device 101 can provide more satisfactory estimation information using genetic information.

As described above, the health management device 101 according to the third example embodiment may acquire, for example, basic information that is information measured by the body composition meter used by the user and that is information related to the body composition of the user. Then, the health management device 101 may calculate the estimation value of the user based on the medical examination information, the effort information, and the basic information using a training model that was machine-trained on the relationship between the change in the value of the examination item of the medical examination, the healthy action, and the basic information.

The health management device 101 according to the third example embodiment may acquire biometric information about the user measured by a measurement instrument installed in a store. Then, the health management device 101 may calculate the estimation value of the user based on the medical examination information, the effort information, and the basic information using a training model that was trained on the relationship between the change in the value of the examination item of the medical examination, the healthy action, the basic information, and the biometric information.

As a result, the health management device 101 can further use the current biometric information about the user such as the basic information, so that the accuracy of calculating the estimation value can be improved.

A person who wants to grasp the health condition may perform a simulation of the health condition. However, in a case where the result of the simulation is output with an index different from that of each of various examination results obtained by the medical examination, it may be difficult for the person to grasp how much the health condition has changed from the result of the past medical examination. When a person cannot grasp a change in health condition, it is difficult for the person to feel an achievement in healthy action. This may lower motivation for healthy action. Reference literature discloses that a health condition is diagnosed, but there is no description of a specific diagnosis result. According to the present disclosure, it is possible to allow a user to grasp a change in health condition.

### Configuration Example of Hardware of Health Management Device

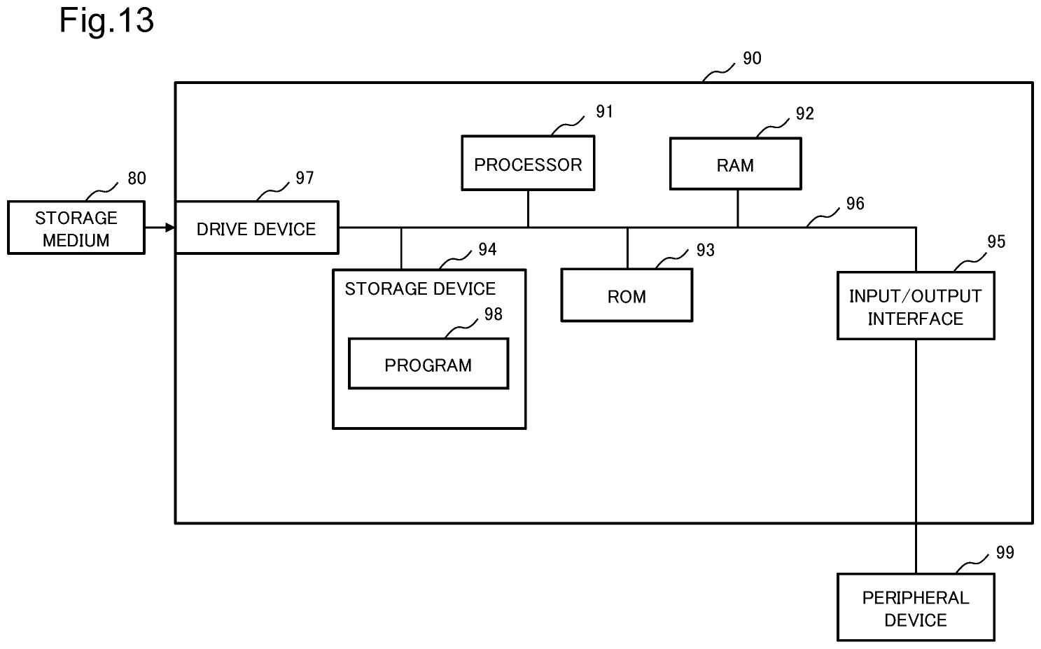

Hardware constituting the above-described health management devices according to the first, second, and third example embodiments will be described. FIG. 13 is a block diagram illustrating an example of a hardware configuration of a computer device constituting the health management device according to each example embodiment. In a computer device 90, the health management device and the health management method described in each example embodiment and each modification are achieved. For example, each of the health management device, the user terminal, and the external server described in each example embodiment and each modification may have the hardware configuration illustrated in FIG. 13. In a case where the health management device and the user terminal are integrated, the integrated device may have the hardware configuration illustrated in FIG. 13.

As illustrated in FIG. 13, the computer device 90 includes a processor 91, a random access memory (RAM) 92, a read only memory (ROM) 93, a storage device 94, an input/output interface 95, a bus 96, and a drive device 97. The health management device may be implemented by a plurality of electric circuits.

The storage device 94 stores a program (computer program) 98. The processor 91 executes the program 98 of the present health management device using the RAM 92. Specifically, for example, the program 98 includes a program that causes a computer to execute the processing illustrated in FIGS. 3, 9, and 12. When the processor 91 executes the program 98, the function of each component of the present health management device is implemented. The program 98 may be stored in the ROM 93. The program 98 may be recorded in a storage medium 80 and read using the drive device 97, or may be transmitted from an external device (not illustrated) to the computer device 90 via a network (not illustrated).

The input/output interface 95 exchanges data with a peripheral device (keyboard, mouse, display device, etc.) 99. The input/output interface 95 functions as a means configured to acquire or outputting data. The bus 96 connects the respective components.

There are various modifications of the method for achieving the health management device. For example, the health management device can be implemented as a dedicated device. The health management device can be implemented based on a combination of a plurality of devices.

A processing method for causing a storage medium to record a program for achieving each configuration in the functions of each example embodiment, reading the program recorded in the storage medium as a code, and a computer executing the program are also included in the scope of each example embodiment. That is, a computer-readable storage medium is also included in the scope of each example embodiment. A storage medium recording the above-described program and the program itself are also included in each example embodiment.

The storage medium is, for example, a floppy (registered trademark) disk, a hard disk, an optical disk, a magneto-optical disk, a compact disc (CD)-ROM, a magnetic tape, a nonvolatile memory card, or a ROM, but is not limited to this example. In addition, the program recorded in the storage medium is not limited to a program that executes processing by itself, and programs that operate on an operating system (OS) to execute processing in cooperation with other software and functions of an extension board are also included in the scope of each example embodiment.

Although the present disclosure is described above with reference to the example embodiments, the present disclosure is not limited to the above example embodiments. It will be understood by those of ordinary skill in the art that various changes in form and details may be made therein without departing from the spirit and scope of the present disclosure as defined by the claims.

The above-described example embodiments and modifications can be appropriately combined.

The previous description of embodiments is provided to enable a person skilled in the art to make and use the present disclosure. Moreover, various modifications to these example embodiments will be readily apparent to those skilled in the art, and the generic principles and specific examples defined herein may be applied to other embodiments without the use of inventive faculty. Therefore, the present disclosure is not intended to be limited to the example embodiments described herein but is to be accorded the widest scope as defined by the limitations of the claims and equivalents.

Further, it is noted that the inventor's intent is to retain all equivalents of the claimed disclosure even if the claims are amended during prosecution.

1. A system for health management comprising:
a user terminal having a display and configured to receive display signal and to provide a predetermined display based on the display signal, the predetermined display including an input form for receiving input of effort information;
one or more memories storing instructions; and
one or more processors in communication with the user terminal and a server of an institution having medical examination information, the processors configured to execute the instructions to:
acquire the medical examination information about a result of a medical examination of a user from the server and the effort information about a healthy action from the user terminal, the healthy action being an action engaged by the user;
calculate an estimation value of an examination item of a medical examination of the user using a training model, the medical examination information and the effort information, the training model being a model trained on a relationship between a change in a value of an examination item of a medical examination and a healthy action by machine learning;
generate advice on a healthy action based on a difference between a value of the examination item indicated in the medical examination information and the estimation value;
convert the calculated estimation value and the advice into the display signal; and
transmit the display signal to the user terminal.

2. The system according to claim 1, wherein
the one or more processors are configured to execute the instruction to present, when there is another user whose medical examination result is similar to a medical examination result of the user, a healthy action being performed by the another user.

3. The system according to claim 1, wherein
the one or more processors are configured to execute the instructions to:
acquire medical examination information and effort information associated with each of a plurality of users;
calculate the estimation value with respect to each of the plurality of users;
calculate a difference between a value of an examination item in the medical examination information and the estimation value for each of two or more users having similar medical examination results;
generate a list in which the two or more users are ranked using the difference; and
present the generated list.

4. The system according to claim 1, wherein
the advice is information about a medium indicating at least one of a method of exercise as a healthy action and content of a meal as a healthy action for assisting the user in decision making.

5. The system according to claim 1, wherein
the one or more processors are configured to execute the instructions to:
acquire information about a place of employment of the user; and
generate the advice including information about a benefit and welfare system related to a healthy action, the benefit and welfare system being a benefit and welfare system of the place of employment of the user.

6. The system according to claim 1, wherein
the one or more processors are configured to execute the instructions to:
acquire basic information, the basic information being information about a body composition of the user measured by a body composition meter; and
calculate the estimation value using a training model, the medical examination information, the effort information, and the basic information, the training model being a model trained on a relationship between a change in a value of an examination item of a medical examination, a healthy action, and basic information.

7. The system according to claim 1, wherein
the one or more processors are configured to execute the instructions to:
acquire biometric information about the user, the biometric information being measured by a measurement instrument installed in a store; and
calculate the estimation value using a training model, the medical examination information, the effort information, and the basic information, the training model being a model trained on a relationship between a change in a value of an examination item of a medical examination, a healthy action, basic information.

8. A method for health management comprising:
by a computer in communication with a user terminal and a server of an institution having medical examination information,
acquiring the medical examination information indicating a result of a medical examination of a user from the server and effort information indicating a healthy action from the user terminal, the healthy action being an action engaged by the user;
calculating an estimation value of an examination item of a medical examination of the user using a training model, the medical examination information and the effort information, the training model being a model trained on a relationship between a change in a value of an examination item of a medical examination and a healthy action by machine learning;
generating advice on a healthy action based on a difference between a value of the examination item indicated in the medical examination information and the estimation value;
converting the calculated estimation value and the advice into display signal; and
transmitting the display signal to the user terminal.

9. A non-transitory computer readable recording medium that records a program for causing a computer, in communication with a user terminal and a server of an institution having medical examination information, to execute:
acquiring the medical examination information indicating a result of a medical examination of a user from the server and effort information indicating a healthy action from the user terminal, the healthy action being an action engaged by the user;
calculating an estimation value of an examination item of a medical examination of the user using a training model, the medical examination information and the effort information, the training model being a model trained on a relationship between a change in a value of an examination item of a medical examination and a healthy action by machine learning;
generating advice on a healthy action based on a difference between a value of the examination item indicated in the medical examination information and the estimation value;
converting the calculated estimation value and the advice into display signal; and
transmitting the display signal to the user terminal.
